(19) United States
(12) Patent Application Publication
(10) Pub. No.: US 2024/0148288 A1
(43) Pub. Date: May 9, 2024

(54) Biological Information Measurement Device And Biological Information Measurement Method

(71) Applicant: SEIKO EPSON CORPORATION, Tokyo (JP)

(72) Inventors: Akira IKEDA, Chino (JP); Tsukasa EGUCHI, Matsumoto (JP)

(21) Appl. No.: 18/504,393

(22) Filed: Nov. 8, 2023

(30) Foreign Application Priority Data
Nov. 9, 2022 (JP) 2022-179371

(51) Int. Cl. A61B 5/1455; A61B 5/024

(52) U.S. Cl. CPC A61B 5/14551; A61B 5/02416; A61B 5/742; A61B 2560/0223

(57) ABSTRACT

A controller of a biological information measurement device performs time-frequency analysis on a first light-receiving signal based on red light, a second light-receiving signal based on infrared light, and a third light-receiving signal based on green light, determines a pulsation band including a frequency of a pulse wave by the time-frequency analysis on the third light-receiving signal, detects a first light-receiving signal intensity of the pulsation band and a second light-receiving signal intensity of the pulsation band, and calculates a fluctuation component ratio between a red light transmitted amount and an infrared light transmitted amount by using the first light-receiving signal intensity and the second light-receiving signal intensity.

The present application is based on, and claims priority from JP Application Serial Number 2022-179371, filed Nov. 9, 2022, the disclosure of which is hereby incorporated by reference herein in its entirety.

### BACKGROUND

### 1. Technical Field

The present disclosure relates to a biological information measurement device and a biological information measurement method.

### 2. Related Art

A measurement device that non-invasively measures biological information of a subject is known. The measurement device described in JP-A-2022-86227 measures a pulse wave and an oxygen saturation concentration. The measurement device includes a first light emitter, a second light emitter, and a third light emitter. The first light emitter emits green light having a green wavelength band to a measurement site. The second light emitter emits red light having a red wavelength band to the measurement site. The third light emitter emits near infrared light having a near infrared wavelength band to the measurement site. The measurement device specifies the pulse wave from a detection signal representing a light-receiving intensity of the green light. The measurement device specifies the oxygen saturation concentration by analyzing a detection signal representing a light-receiving intensity of the red light and a detection signal representing a light-receiving intensity of the near infrared light. The measurement device specifies the oxygen saturation concentration using a pulsation component of an artery.

Due to the influence of body movement of the subject, an outside air temperature, or the like, it is difficult to detect a pulse wave component based on the red light or infrared light. When it is difficult to detect the pulse wave component, a measurement accuracy of a fluctuation component ratio of an absorbed light amount used for determining the oxygen saturation concentration is decreased, and a measure for the decrease in the measurement accuracy is not sufficiently performed.

### SUMMARY

A biological information measurement device according to the present disclosure includes: a light-emitting unit including a first light-emitting element that emits red light, a second light-emitting element that emits infrared light, and a third light-emitting element that emits green light; a light-receiving unit configured to receive the red light, the infrared light, and the green light emitted by the light-emitting unit, and generate a first light-receiving signal based on the red light, a second light-receiving signal based on the infrared light, and a third light-receiving signal based on the green light; and a controller configured to calculate biological information based on the first light-receiving signal, the second light-receiving signal, and the third light-receiving signal, in which the controller is configured to perform time-frequency analysis on the first light-receiving signal, the second light-receiving signal, and the third light-receiving signal, determine a pulsation band including a frequency of a pulse wave by the time-frequency analysis on the third light-receiving signal, detect a first light-receiving signal intensity of the pulsation band and a second light-receiving signal intensity of the pulsation band, and calculate a fluctuation component ratio between a red light transmitted amount and an infrared light transmitted amount by using the first light-receiving signal intensity and the second light-receiving signal intensity.

A biological information measurement method according to the present disclosure includes: emitting red light, infrared light, and green light to a subject; receiving the red light, the infrared light, and the green light passing through the subject; generating a first light-receiving signal based on the received red light, a second light-receiving signal based on the received infrared light, and a third light-receiving signal based on the received green light; performing time-frequency analysis on the first light-receiving signal, the second light-receiving signal, and the third light-receiving signal; determining a pulsation band including a frequency of a pulse wave by the time-frequency analysis on the third light-receiving signal; detecting a first light-receiving signal intensity of the pulsation band and a second light-receiving signal intensity of the pulsation band; and calculating a fluctuation component ratio between a red light transmitted amount and an infrared light transmitted amount by using the first light-receiving signal intensity and the second light-receiving signal intensity.

### BRIEF DESCRIPTION OF THE DRAWINGS

FIG. 1 is a diagram showing a schematic configuration of a measurement device.

FIG. 2 is a diagram showing a schematic configuration of a measurement surface.

FIG. 3 is a diagram showing a block configuration of the measurement device.

FIG. 4 is a diagram schematically showing a detection signal.

FIG. 5A is a diagram showing a change with time of a red light detection signal.

FIG. 5B is a diagram showing a red light spectrogram.

FIG. 6A is a diagram showing a change with time of an infrared light detection signal.

FIG. 6B is a diagram showing an infrared light spectrogram.

FIG. 7A is a diagram showing a change with time of a green light detection signal.

FIG. 7B is a diagram showing a green light spectrogram.

FIG. 8 is a diagram showing a relationship between a frequency and a signal intensity of each detection signal in a predetermined time.

FIG. 9 is a flowchart for calculating a fluctuation component amplitude ratio.

FIG. 10 is a flowchart for determining an oxygen saturation concentration.

### DESCRIPTION OF EMBODIMENTS

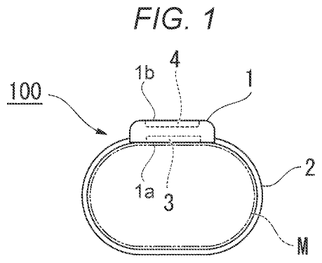

FIG. 1 shows a schematic configuration of a measurement device 100. FIG. 1 is a side view of the measurement device 100. The measurement device 100 non-invasively measures biological information of a user M such as a human. The measurement device 100 is a watch type portable device worn on a measurement site of the user M. The measurement device 100 shown in FIG. 1 is worn on, for example, the wrist of the user M.

The measurement device 100 measures the biological information of the user M. The measurement device 100 measures a pulse wave such as a pulse interval and an oxygen saturation concentration as the biological information. The pulse wave interval is represented as a post pacing interval (PPI). The oxygen saturation concentration is represented as SpO₂. The pulse wave indicates a change with time in a volume of a blood vessel in conjunction with the heart beat. The oxygen saturation concentration indicates a proportion of hemoglobin bound to oxygen to hemoglobin in arterial blood of the user M. The oxygen saturation concentration is an index for evaluating a breathing function of the user M. The measurement device 100 may measure the biological information other than the pulse and the oxygen saturation concentration. The measurement device 100 measures, for example, a glucose concentration in the arterial blood and an alcohol concentration in the arterial blood. The measurement device 100 corresponds to an example of a biological information measurement device. The user M corresponds to an example of a subject. The measurement device 100 includes a housing 1 and a belt 2. The housing 1 accommodates a detection unit 3 and a display panel 4.

The housing 1 is an exterior housing accommodating a unit or the like provided in the measurement device 100. The housing 1 has a measurement surface 1a and a display surface 1b. The measurement surface 1a is a surface facing the measurement site of the user M. The measurement surface 1a comes into contact with the measurement site of the user M. The display surface 1b is a surface visible to the user M. In addition to the detection unit 3 and the display panel 4, the housing 1 accommodates a control unit 30, a memory 40, or the like, which will be described later.

The belt 2 is a member that is used when the user M wears the housing 1 on the measurement site. The belt 2 is attached to, for example, a side surface of the housing 1. The belt 2 is wound around the measurement site, and thereby the housing 1 is worn on the measurement site of the user M.

The detection unit 3 is disposed on the measurement surface 1a of the housing 1. The detection unit 3 is disposed at a position facing the measurement site of the user M. The detection unit 3 acquires various types of information used when measuring the biological information.

The display panel 4 is disposed on the display surface 1b of the housing 1. The display panel 4 is visible to the user M. The display panel 4 displays various types of the measured biological information. The display panel 4 may display various types of information such as time other than the biological information. The display panel 4 corresponds to an example of a display unit.

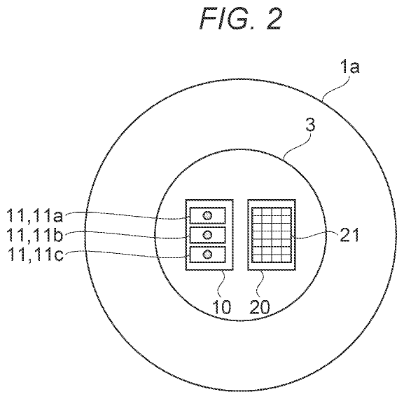

FIG. 2 shows a schematic configuration of the measurement surface 1a. FIG. 2 shows the schematic configuration of the measurement surface 1a when viewed from the outside. The measurement surface 1a shown in FIG. 2 is formed in a circular shape, but is not limited thereto. The measurement surface 1a may be formed in various shapes such as a square shape and an elliptical shape. The detection unit 3 is disposed on the measurement surface 1a. The detection unit 3 includes a light-emitting element unit 10 and a light-receiving element unit 20.

The light-emitting element unit 10 emits light toward the measurement site of the user M. The light-emitting element unit 10 includes a plurality of light-emitting elements 11. The light-emitting element unit 10 shown in FIG. 2 includes a red light-emitting element 11a, an infrared light-emitting element 11b, and a green light-emitting element 11c. The number of light-emitting elements 11 is not limited to three. Four or more light-emitting elements 11 may be provided on the light-emitting element unit 10. The light-emitting element unit 10 corresponds to an example of a light-emitting unit.

The light-emitting element 11 is implemented with a bare chip type or a shell type light emitting diode (LED). The light-emitting element 11 may be implemented with a laser diode. A configuration of the light-emitting element 11 is appropriately set according to a wavelength range of emitted light.

The red light-emitting element 11a emits red light RL in a wavelength range of 600 nm to 800 nm toward the measurement site. The red light RL is, for example, light having a peak wavelength of 660 nm. The red light-emitting element 11a corresponds to an example of a first light-emitting element.

The infrared light-emitting element 11b emits infrared light NL in a wavelength range of 800 nm to 1300 nm toward the measurement site. The infrared light NL is, for example, near infrared light having a peak wavelength of 905 nm. The infrared light-emitting element 11b corresponds to an example of a second light-emitting element.

The green light-emitting element 11c emits green light GL in a wavelength range of 520 nm to 550 nm toward the measurement site. The green light GL is, for example, light having a peak wavelength of 520 nm. The green light-emitting element 11c corresponds to an example of a third light-emitting element.

In the light-emitting element unit 10 shown in FIG. 2, the red light-emitting element 11a, the infrared light-emitting element 11b, and the green light-emitting element 11c are disposed in this order, but the order is not limited thereto. The red light-emitting element 11a, the infrared light-emitting element 11b, and the green light-emitting element 11c can be appropriately disposed in the light-emitting element unit 10.

The light-receiving element unit 20 receives various types of light emitted by the light-emitting element unit 10. The light-receiving element unit 20 includes a light-receiving element 21 that receives various types of light. The light-receiving element 21 receives the red light RL, the infrared light NL, and the green light GL emitted by the light-emitting element unit 10. The light-receiving element 21 includes one or a plurality of photodiodes. The light-receiving element unit 20 corresponds to an example of a light-receiving unit.

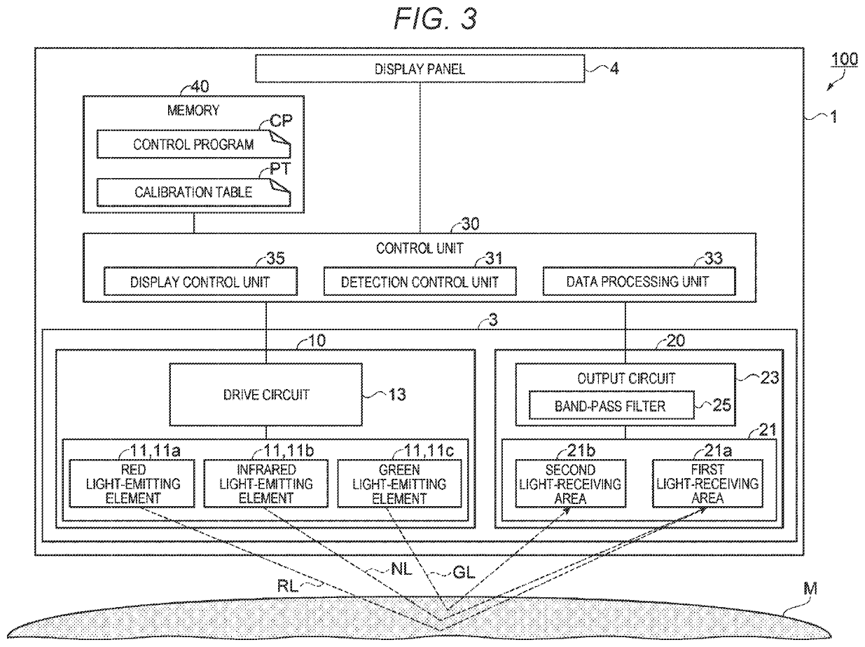

FIG. 3 shows a block configuration of the measurement device 100. FIG. 3 shows the measurement device 100 excluding the belt 2. The measurement device 100 accommodates various units or the like in the housing 1. The measurement device 100 includes the detection unit 3, the control unit 30, the memory 40, and the display panel 4.

The detection unit 3 is an optical sensor module that detects data related to the biological information using light of various wavelength ranges as a detection signal. The detection unit 3 includes the light-emitting element unit 10 and the light-receiving element unit 20.

The light-emitting element unit 10 includes the plurality of light-emitting elements 11 and a drive circuit 13. The plurality of light-emitting elements 11 shown in FIG. 3 are the red light-emitting element 11a, the infrared light-emitting element 11b, and the green light-emitting element 11c. The red light-emitting element 11a emits the red light RL toward the measurement site of the user M. The infrared light-emitting element 11b emits the infrared light NL toward the measurement site of the user M. The green light-emitting element 11c emits the green light GL toward the measurement site of the user M.

The drive circuit 13 drives the plurality of light-emitting elements 11. The drive circuit 13 causes the plurality of light-emitting elements 11 to emit light under the control of the control unit 30. The drive circuit 13 causes the red light-emitting element 11a, the infrared light-emitting element 11b, and the green light-emitting element 11c to emit light.

The light-receiving element unit 20 includes the light-receiving element 21 and an output circuit 23. The light-receiving element 21 receives light emitted by the light-emitting element 11 and reflected by the measurement site of the user M. The light-receiving element 21 receives the red light RL, the infrared light NL, and the green light GL reflected by the measurement site of the user M. The light-receiving element 21 is divided into a plurality of regions. The light-receiving element 21 may be divided into the plurality of regions using an optical filter (not shown). The light-receiving element 21 shown in FIG. 3 is divided into a first light-receiving area 21a and a second light-receiving area 21b.

The first light-receiving area 21a receives the red light RL and the infrared light NL. The first light-receiving area 21a receives the red light RL emitted by the red light-emitting element 11a and reflected by the measurement site of the user M. The first light-receiving area 21a receives the infrared light NL emitted by the infrared light-emitting element 11b and reflected by the measurement site of the user M. The first light-receiving area 21a may receive at least one of the red light RL and the infrared light NL through the optical filter. The first light-receiving area 21a may alternately receive the red light RL and the infrared light NL in a time-division manner.

The second light-receiving area 21b receives the green light GL. The second light-receiving area 21b receives the green light GL emitted by the green light-emitting element 11c and reflected by the measurement site of the user M. The second light-receiving area 21b may receive the green light GL via the optical filter.

In FIG. 3, the red light RL and the infrared light NL are received by the first light-receiving area 21a, but are not limited thereto. A third light-receiving area different from the first light-receiving area 21a and the second light-receiving area 21b may be provided. The red light RL or the infrared light NL may be received by the third light-receiving area. At this time, when the third light-receiving area receives the infrared light NL, the first light-receiving area 21a receives the red light RL. The light-receiving element 21 may not be divided into the plurality of regions. The light-receiving element 21 may receive the red light RL, the infrared light NL, and the green light GL in a time-division manner.

The light-receiving element unit 20 shown in FIG. 3 receives reflected light of the red light RL and reflected light of the infrared light NL, but is not limited thereto. The light-receiving element unit 20 may receive the red light RL transmitted through the user M and the infrared light NL transmitted through the user M. The light-receiving element unit 20 receives transmitted light of the red light RL and transmitted light of the infrared light NL.

The output circuit 23 outputs, to the control unit 30, a detection signal based on the light received by the light-receiving element 21. The output circuit 23 generates a detection signal by performing processing such as analog-to-digital conversion on light-receiving intensity data of the light received by the light-receiving element 21. The output circuit 23 generates a red light detection signal based on the red light RL received by the first light-receiving area 21a. The output circuit 23 generates an infrared light detection signal based on the infrared light NL received by the first light-receiving area 21a. The output circuit 23 generates a green light detection signal based on the green light GL received by the second light-receiving area 21b. The red light detection signal corresponds to an example of a first light-receiving signal. The infrared light detection signal corresponds to an example of a second light-receiving signal. The green light detection signal corresponds to an example of a third light-receiving signal.

The output circuit 23 may include a band-pass filter 25. The band-pass filter 25 separates an AC component and a DC component from the light-receiving intensity data. The band-pass filter 25 separates the light-receiving intensity data into an AC component and a DC component by extracting the AC component from the light-receiving intensity data. The band-pass filter 25 outputs the separated AC component and the DC component as detection signals to the control unit 30.

The control unit 30 is a controller that controls operations of various units. The control unit 30 is, for example, a processor including a central processing unit (CPU). The control unit 30 may include one or a plurality of processors. The control unit 30 may include a semiconductor memory such as a RAM (random access memory) or a read only memory (ROM). The semiconductor memory functions as a work area of the control unit 30. The control unit 30 functions as a detection control unit 31, a data processing unit 33, and a display control unit 35 by executing a control program CP stored in the memory 40. The control unit 30 corresponds to an example of a controller.

The detection control unit 31 is a functional unit that operates in the control unit 30. The detection control unit 31 controls the light-emitting element unit 10 and the light-receiving element unit 20. The detection control unit 31 adjusts a light-emitting timing, a light extinction timing, a light amount, or the like of the light-emitting element 11 via the drive circuit 13. The detection control unit 31 controls a light-receiving timing, a light-receiving time, digital-to-analog conversion, or the like of various types of light for the light-receiving element unit 20. The detection control unit 31 may separate the AC component and the DC component from the light-receiving intensity data by controlling the band-pass filter 25.

The data processing unit 33 is a functional unit that operates in the control unit 30. The data processing unit 33 processes the detection signal output from the light-receiving element unit 20. The data processing unit 33 acquires the red light detection signal, the infrared light detection signal, and the green light detection signal from the light-receiving element unit 20.

The data processing unit 33 performs short time Fourier transform on the detection signal. The data processing unit 33 analyzes frequency information that changes with time by performing the short time Fourier transform on the detection signal. The data processing unit 33 obtains a spectrogram in a predetermined frequency range by performing the short time Fourier transform on the detection signal. The predetermined frequency range is a range including the frequency of the pulse wave. The predetermined frequency range is, for example, a range of 0.5 Hz to 2 Hz. The predetermined frequency range is appropriately adjusted according to the wavelength range of the light subjected to the short time Fourier transform. The data processing unit 33 performs the short time Fourier transform on the red light detection signal to obtain a red light spectrogram. The data processing unit 33 performs the short time Fourier transform on the infrared light detection signal to obtain an infrared light spectrogram. The data processing unit 33 performs the short time Fourier transform on the green light detection signal to obtain a green light spectrogram. Details of the red light spectrogram, the infrared light spectrogram, and the green light spectrogram will be described later. The data processing unit 33 corresponds to an example of a controller.

The data processing unit 33 performs the short time Fourier transform on the detection signal, but is not limited to the short time Fourier transform. The method is not limited as long as the frequency information that changes with time for the detection signal can be analyzed. The data processing unit 33 may perform, for example, wavelet conversion. The short time Fourier transform corresponds to an example of time-frequency analysis.

The data processing unit 33 determines a pulse wave region PB using the green light spectrogram. The pulse wave region PB is a region including the frequency of the pulse wave. The pulse wave region PB is a frequency region including the frequency of the pulse wave for each time. The pulse wave region PB is, for example, a region including a peak value in the green light detection signal. The pulse wave region PB corresponds to an example of a pulsation band. The green light detection signal is less susceptible to disturbance caused by body movement or the like than the red light detection signal and the infrared light detection signal. The data processing unit 33 can specify the frequency of the pulse wave with a high measurement accuracy by determining the pulse wave region PB using the green light spectrogram.

The data processing unit 33 detects a red light detection signal intensity of the pulse wave region PB and an infrared light detection signal intensity of the pulse wave region PB. The red light detection signal intensity represents a signal intensity of the red light detection signal in the pulse wave region PB. The red light detection signal intensity is, for example, a peak value of the red light detection signal in the pulse wave region PB. The infrared light detection signal intensity represents a signal intensity of the infrared light detection signal in the pulse wave region PB. The infrared light detection signal intensity is, for example, a peak value of the infrared light detection signal in the pulse wave region PB. The red light detection signal intensity corresponds to an example of a first light-receiving signal intensity. The infrared light detection signal intensity corresponds to an example of a second light-receiving signal intensity.

The data processing unit 33 calculates a fluctuation component amplitude ratio using the red light detection signal intensity and the infrared light detection signal intensity. The fluctuation component amplitude ratio is a ratio between the red light transmitted amount and the infrared light transmitted amount. The red light transmitted amount is a light amount of the red light RL, which is emitted from the red light-emitting element 11a, is transmitted through the measurement site of the user M, and reaches the light-receiving element 21. The infrared light transmitted amount is a light amount of the infrared light NL, which is emitted from the infrared light-emitting element 11b, is transmitted through the measurement site of the user M, and reaches the light-receiving element 21. The fluctuation component amplitude ratio corresponds to an example of a fluctuation component ratio. The fluctuation component amplitude ratio is calculated by the following formula (1).

R=(AC_{Red}/DC_{Red})/(AC_{IR}/DC_{IR}) (1)

Here, R indicates the fluctuation component amplitude ratio. AC_{Red} indicates an intensity of an AC component of the red light detection signal. DC_{Red} indicates an intensity of a DC component of the red light detection signal. AC_{IR} indicates an intensity of an AC component of the infrared light detection signal. DC_{IR} indicates an intensity of a DC component of the infrared light detection signal.

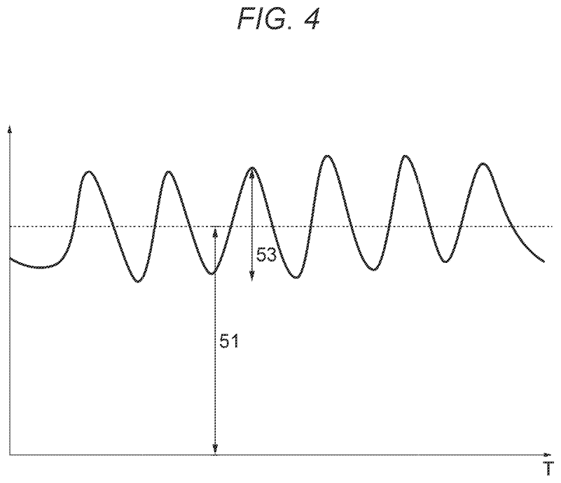

FIG. 4 schematically shows a detection signal. A horizontal axis in FIG. 4 indicates time. A vertical axis in FIG. 4 indicates an intensity of the detection signal. FIG. 4 schematically shows an example of the detection signal output from the output circuit 23.

The detection signal is data of a signal intensity detected at a predetermined interval. The signal intensity is detected, for example, n times per second. n is an integer of 1 or more. The signal intensity includes DC component data 51 as a DC component and AC component data 53 as an AC component. The data processing unit 33 separates the DC component data 51 and the AC component data 53 from the signal intensity, and calculates the fluctuation component amplitude ratio. The DC component data 51 and the AC component data 53 may be separated by the band-pass filter 25 provided in the output circuit 23. The band-pass filter 25 generates the DC component data 51 and the AC component data 53 by extracting the AC component data 53 from the light-receiving intensity data. The output circuit 23 may output the DC component data 51 and the AC component data 53 as detection signals to the control unit 30.

The data processing unit 33 may determine the oxygen saturation concentration based on the calculated fluctuation component amplitude ratio. The data processing unit 33 refers to a calibration table PT stored in the memory 40 to obtain a value of the oxygen saturation concentration corresponding to the calculated fluctuation component amplitude ratio. The data processing unit 33 determines, as the oxygen saturation concentration, the value of the oxygen saturation concentration corresponding to the fluctuation component amplitude ratio. The data processing unit 33 outputs the oxygen saturation concentration to the display control unit 35. The data processing unit 33 may output the oxygen saturation concentration to an external device via a communication interface (not shown).

The data processing unit 33 may determine the oxygen saturation concentration at a predetermined time interval and calculate a moving average of a plurality of oxygen saturation concentrations determined at the predetermined time interval. For example, the data processing unit 33 determines the oxygen saturation concentration per second. The data processing unit 33 calculates the moving average of the oxygen saturation concentrations per second. The data processing unit 33 outputs the calculated moving average of the oxygen saturation concentrations to the display control unit 35 or the communication interface. The data processing unit 33 calculates the moving average of the oxygen saturation concentrations, but is not limited thereto. The data processing unit 33 may calculate a moving average of fluctuation component amplitude ratios calculated at a predetermined time interval. The data processing unit 33 determines the oxygen saturation concentration based on the moving average of the fluctuation component amplitude ratios.

The display control unit 35 is a functional unit that operates in the control unit 30. The display control unit 35 controls display of the display panel 4. The display control unit 35 causes the display panel 4 to display various images by transmitting display data to the display panel 4.

The display control unit 35 acquires the oxygen saturation concentration from the data processing unit 33. The display control unit 35 generates the display data including the oxygen saturation concentration. The display control unit 35 outputs the display data including the oxygen saturation concentration to the display panel 4. The display control unit 35 causes the display panel 4 to display the oxygen saturation concentration based on the display data. The display control unit 35 may cause the display panel 4 to display the moving average of the oxygen saturation concentrations based on the display data including the moving average of the oxygen saturation concentrations. The display control unit 35 corresponds to an example of a controller.

The memory 40 stores various types of data. The memory 40 stores control data for operating various units, various types of data calculated by the control unit 30, or the like. The memory 40 may store the oxygen saturation concentration or the like determined by the data processing unit 33. The memory 40 stores the control program CP that operates in the control unit 30. The memory 40 stores the calibration table PT referred to by the data processing unit 33. The memory 40 includes a ROM, a RAM, or the like. The memory 40 corresponds to an example of a storage unit.

The control program CP is executed by the control unit 30 to operate various functional units. The control program CP causes the control unit 30 to operate as the detection control unit 31, the data processing unit 33, and the display control unit 35. The control program CP may cause the control unit 30 to operate as a functional unit other than the detection control unit 31, the data processing unit 33, and the display control unit 35.

The calibration table PT is a table that stores the fluctuation component amplitude ratio and the oxygen saturation concentration in association with each other. The calibration table PT shows a relationship between the fluctuation component amplitude ratio and the oxygen saturation concentration. The calibration table PT is created in advance by a manufacturer of the measurement device 100. The data processing unit 33 can determine the oxygen saturation concentration corresponding to the calculated fluctuation component amplitude ratio by referring to the calibration table PT. The calibration table PT corresponds to an example of a calibration curve table.

The memory 40 may store a calibration formula instead of the calibration table PT. The calibration formula is a relational expression between the fluctuation component amplitude ratio and the oxygen saturation concentration. The data processing unit 33 calculates the oxygen saturation concentration corresponding to the calculated fluctuation component amplitude ratio using the calibration formula.

The display panel 4 displays various images. The display panel 4 displays the oxygen saturation concentration under the control of the display control unit 35. The display panel 4 displays the oxygen saturation concentration based on the display data output from the display control unit 35. The display panel 4 may display a pulse rate or the like. The pulse rate is measured based on, for example, the green light detection signal. The display panel 4 includes a liquid crystal display, an organic electro-luminescence (EL) display, or the like.

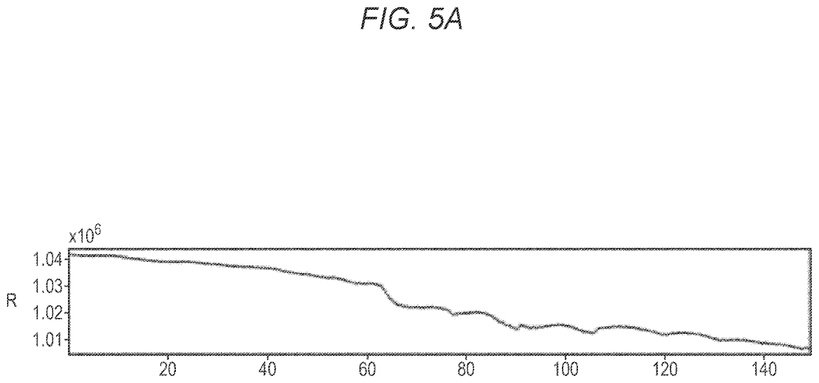

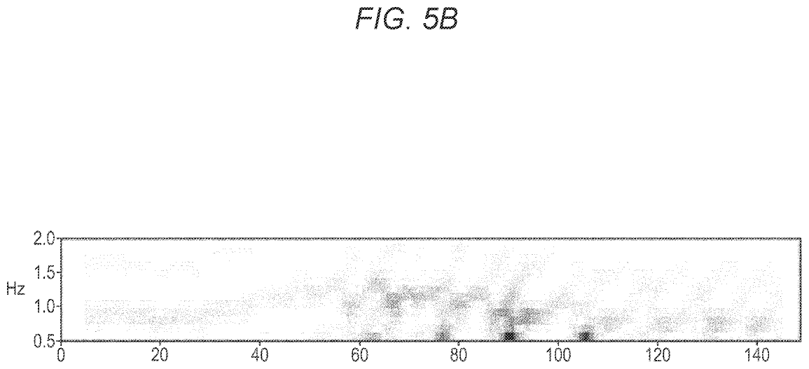

FIGS. 5A, 5B, 6A, 6B, 7A, and 7B each show a detection signal of a color or a spectrogram of a detection signal. FIGS. 5A, 5B, 6A, 6B, 7A, and 7B show data based on light received by the light-receiving element unit 20. In FIGS. 5A, 6A, and 7A, the horizontal axis indicates time. In FIGS. 5A, 6A, and 7A, the vertical axis indicates a signal intensity. The horizontal axes in FIGS. 5B, 6B, and 7B indicate time. The vertical axes in FIGS. 5B, 6B, and 7B indicate frequency. Color densities in FIGS. 5B, 6B, and 7B indicate signal intensities.

FIGS. 5A and 5B show data related to the red light RL received by the light-receiving element unit 20. FIG. 5A shows a change with time of the red light detection signal. FIG. 5B shows the red light spectrogram. FIG. 5B shows the red light spectrogram when the short time Fourier transform is performed on the red light detection signal.

The red light detection signal shown in FIG. 5A includes the DC component data 51 of the red light RL and the AC component data 53 of the red light RL. The data processing unit 33 performs the short time Fourier transform on the red light detection signal shown in FIG. 5A to calculate the red light spectrogram shown in FIG. 5B.

FIG. 5B shows a frequency at which the signal intensity is high at each time. In a region of 0 seconds to 40 seconds in FIG. 5B, the signal intensity at the frequency around 1.0 Hz is higher than the signal intensities at the other frequencies. The frequency around 1.0 Hz is a pulsation component. At 90 seconds and 105 seconds in FIG. 5B, the signal intensity at the frequency in the vicinity of 0.5 Hz is higher than the signal intensities at the other frequencies. The signal intensity at the frequency in the vicinity of 0.5 Hz increases discretely, and is presumed to be an influence of disturbance caused by body movement or the like. FIG. 5B shows that there is a time region in which the pulsation component can be detected but it is difficult to detect the pulsation component due to the influence of disturbance caused by body movement or the like.

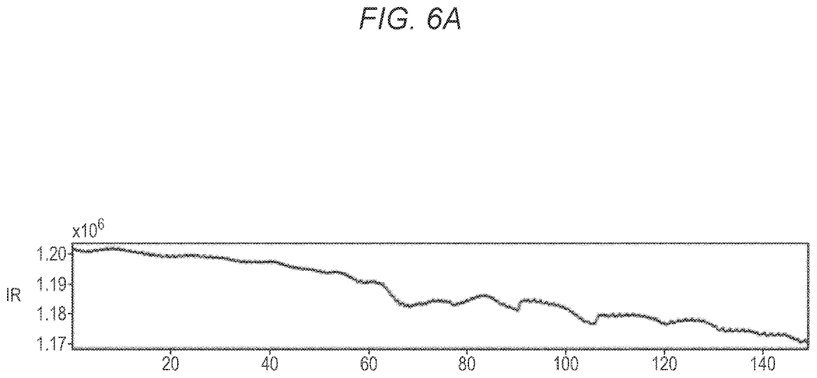

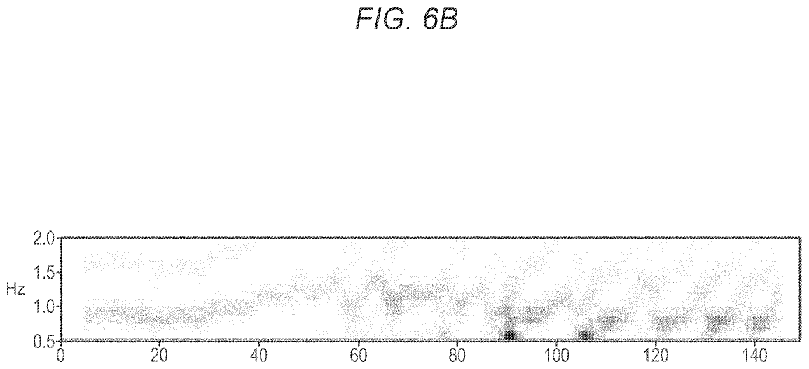

FIGS. 6A and 6B show data related to the infrared light NL received by the light-receiving element unit 20. FIG. 6A shows a change with time of the infrared light detection signal. FIG. 6B shows the infrared light spectrogram. FIG. 6B shows the infrared light spectrogram when the short time Fourier transform is performed on the infrared light detection signal.

The infrared light detection signal shown in FIG. 6A includes the DC component data 51 of the infrared light NL and the AC component data 53 of the infrared light NL. The data processing unit 33 performs the short time Fourier transform on the infrared light detection signal shown in FIG. 6A to calculate the infrared light spectrogram shown in FIG. 6B.

FIG. 6B shows a frequency at which the signal intensity is high at each time. In a region of 0 seconds to 40 seconds in FIG. 6B, the signal intensity at the frequency around 1.0 Hz is higher than the signal intensities at the other frequencies. The frequency around 1.0 Hz is a pulsation component. At 90 seconds and 105 seconds in FIG. 6B, as in FIG. 5B, the signal intensity at the frequency in the vicinity of 0.5 Hz is higher than the signal intensities at the other frequencies. The signal intensity at the frequency in the vicinity of 0.5 Hz increases discretely, and is presumed to be an influence of disturbance caused by body movement or the like. FIG. 6B shows that there is a time region in which the pulsation component can be detected but it is difficult to detect the pulsation component due to the influence of disturbance caused by body movement or the like.

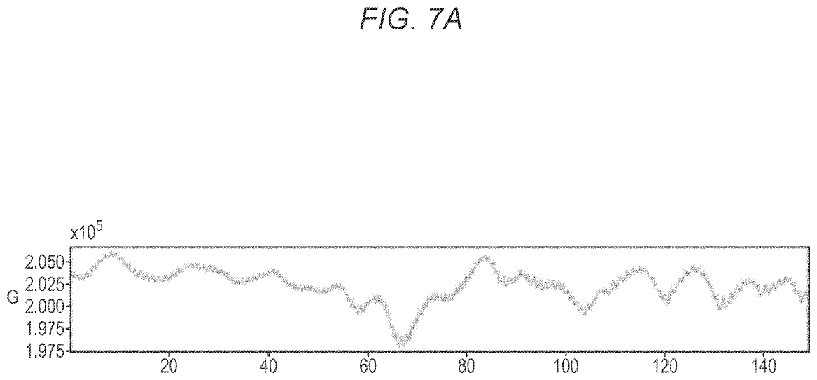

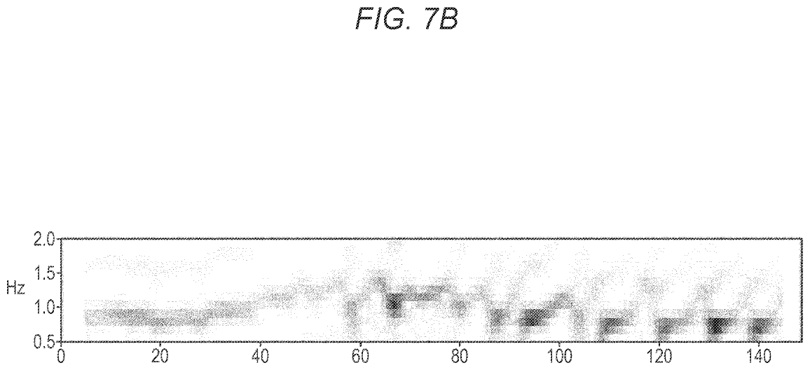

FIGS. 7A and 7B show data related to the green light GL received by the light-receiving element unit 20. FIG. 7A shows a change with time of the green light detection signal. FIG. 7B shows the green light spectrogram. FIG. 7B shows the green light spectrogram when the short time Fourier transform is performed on the green light detection signal.

The green light detection signal shown in FIG. 7A includes the DC component data 51 of the green light GL and the AC component data 53 of the green light GL. The data processing unit 33 performs the short time Fourier transform on the green light detection signal shown in FIG. 7A to calculate the green light spectrogram shown in FIG. 7B.

FIG. 7B shows a frequency at which the signal intensity is high at each time. FIG. 7B shows that the signal intensity at the frequency around 1.0 Hz is higher than the signal intensities at the other frequencies in the entire time region. The frequency around 1.0 Hz is a pulsation component. In FIG. 7B, the signal intensity at the frequency in the vicinity of 0.5 Hz is not higher than the signal intensities at the other frequencies at 90 seconds and 105 seconds as in FIGS. 5B and 6B. FIG. 7B shows that there is no influence of disturbance caused by body movement or the like. FIG. 7B shows that the green light detection signal easily extracts the pulsation component. The data processing unit 33 can easily detect the pulse wave region PB by using the green light detection signal.

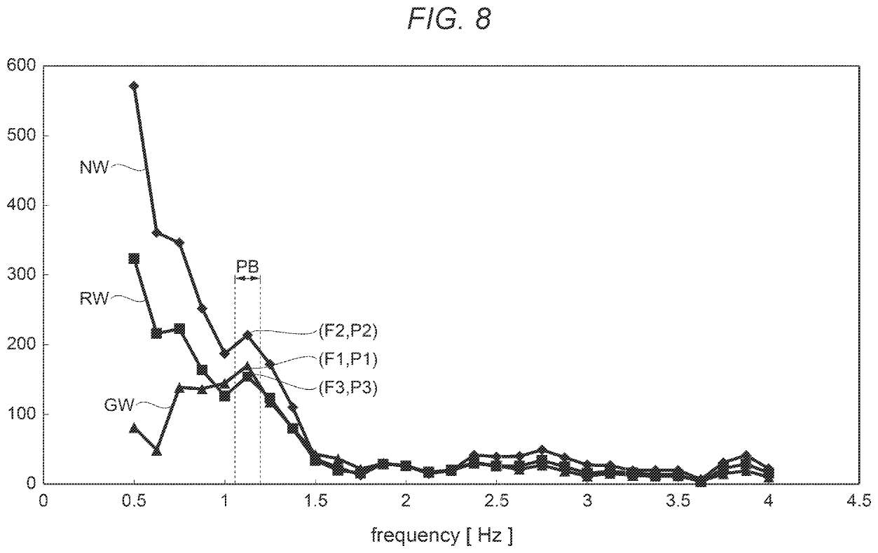

FIG. 8 shows a relationship between a frequency and a signal intensity of each detection signal in a predetermined time. FIG. 8 shows red light data RW, infrared light data NW, and green light data GW in the predetermined time. The predetermined time is, for example, 90 seconds shown in FIGS. 5B, 6B, and 7B. The red light data RW shown in FIG. 8 indicates a relationship between a frequency and a signal intensity of the red light RL when an elapsed time is 90 seconds. The infrared light data NW shown in FIG. 8 indicates a relationship between a frequency and a signal intensity of the infrared light NL when the elapsed time is 90 seconds. The green light data GW shown in FIG. 8 indicates a relationship between a frequency and a signal intensity of the green light GL when the elapsed time is 90 seconds. The relationship between the frequency and the signal intensity of each detection signal can be plotted for each time.

The red light data RW shown in FIG. 8 has a signal intensity higher in a frequency range of 0.5 Hz to 1.0 Hz than in other ranges. The red light data RW indicates a first signal P1 that is a peak value of a signal intensity at a first frequency F1 in the vicinity of 1.1 Hz.

The infrared light data NW shown in FIG. 8 has a signal intensity higher in a frequency range of 0.5 Hz to 1.0 Hz than in other ranges. The infrared light data NW indicates a second signal P2 that is a peak value of a signal intensity at a second frequency F2 in the vicinity of 1.1 Hz.

The green light data GW shown in FIG. 8 does not indicate a signal intensity higher in a frequency range of 0.5 Hz to 1.0 Hz than in other ranges. The green light data GW indicates a third signal P3 that is a peak value of a signal intensity at a third frequency F3 in the vicinity of 1.1 Hz.

The red light data RW and the infrared light data NW indicate signal intensities higher in a frequency range of 0.5 Hz to 1.0 Hz than in other ranges. The green light data GW does not indicate a signal intensity higher in a frequency range of 0.5 Hz to 1.0 Hz than in other ranges. Since the green light spectrogram is less susceptible to disturbance caused by body movement or the like, it can be determined that the signal intensity in the frequency range of 0.5 Hz to 1.0 Hz is affected by disturbance caused by body movement or the like.

The green light data GW indicates the third signal P3 that is the peak value of the signal intensity at the third frequency F3. When the predetermined time is 90 seconds, the pulse wave component can be determined to be the third frequency F3. The data processing unit 33 determines a region including the third frequency F3 as the pulse wave region PB. The data processing unit 33 determines a frequency range including the third frequency F3 as the pulse wave region PB. A width of the frequency range is appropriately set. For example, the data processing unit 33 sets a predetermined frequency range centered on the third frequency F3 as the pulse wave region PB.

The data processing unit 33 detects the red light detection signal intensity included in the pulse wave region PB. For example, when the first frequency F1 is included in the pulse wave region PB, the data processing unit 33 detects the first signal P1, which is the signal intensity at the first frequency F1, as the red light detection signal intensity. The data processing unit 33 may calculate, as the red light detection signal intensity, an added value of the first signal P1 at the first frequency F1 and a signal value at a frequency adjacent to the first frequency F1. The data processing unit 33 may detect, as the red light detection signal intensity, a sum of the red light detection signals included in the pulse wave region PB. The data processing unit 33 uses the detected red light detection signal intensity to calculate the fluctuation component amplitude ratio.

The data processing unit 33 detects the infrared light detection signal intensity included in the pulse wave region PB. For example, when the second frequency F2 is included in the pulse wave region PB, the data processing unit 33 detects the second signal P2, which is the signal intensity at the second frequency F2, as the infrared light detection signal intensity. The data processing unit 33 may calculate, as the infrared light detection signal intensity, an added value of the second signal P2 at the second frequency F2 and a signal value of a frequency adjacent to the second frequency F2. The data processing unit 33 may detect, as the infrared light detection signal intensity, a sum of the infrared light detection signals included in the pulse wave region PB. The data processing unit 33 uses the detected infrared light detection signal intensity to calculate the fluctuation component amplitude ratio.

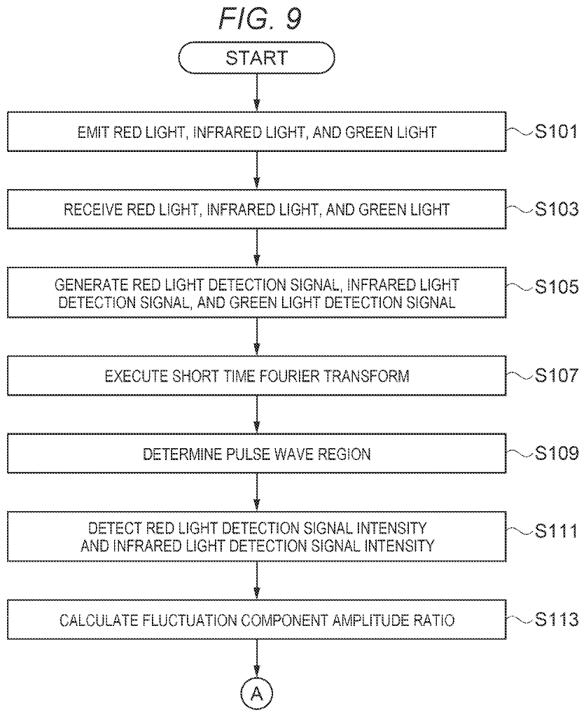

FIG. 9 shows a flowchart for calculating the fluctuation component amplitude ratio. The flowchart shown in FIG. 9 shows an oxygen saturation concentration measurement method. The oxygen saturation concentration measurement method corresponds to an example of a biological information measurement method. FIG. 9 shows a method of calculating the fluctuation component amplitude ratio performed by the measurement device 100.

In step S101, the measurement device 100 emits the red light RL, the infrared light NL, and the green light GL. The detection control unit 31 operated in the control unit 30 causes, via the drive circuit 13, the light-emitting element 11 to emit light. The detection control unit 31 controls the light-emitting element unit 10 to emit light to the user M. The detection control unit 31 causes the light-emitting element unit 10 to emit the red light RL to the user M. The red light-emitting element 11a emits the red light RL to the user M. The detection control unit 31 causes the light-emitting element unit 10 to emit the infrared light NL to the user M. The infrared light-emitting element 11b emits the infrared light NL to the user M. The detection control unit 31 causes the light-emitting element unit 10 to emit the green light GL to the user M. The green light-emitting element 11c emits the green light GL to the user M.

After emitting the red light RL, the infrared light NL, and the green light GL, the measurement device 100 receives the red light RL, the infrared light NL, and the green light GL in step S103. The detection control unit 31 causes the light-receiving element unit 20 to receive the red light RL, the infrared light NL, and the green light GL that passed through the user M. The light-receiving element 21 of the light-receiving element unit 20 receives the red light RL, the infrared light NL, and the green light GL reflected by the user M. The first light-receiving area 21a of the light-receiving element unit 20 receives the red light RL and the infrared light NL. The second light-receiving area 21b receives the green light GL.

After receiving the red light RL, the infrared light NL, and the green light GL, the measurement device 100 generates the red light detection signal, the infrared light detection signal, and the green light detection signal in step S105. The output circuit 23 of the light-receiving element unit 20 generates the red light detection signal based on the received red light RL. The output circuit 23 generates the red light detection signal by performing processing such as analog-to-digital conversion on the light-receiving intensity data of the red light RL detected by the light-receiving element 21. The output circuit 23 generates the infrared light detection signal based on the received infrared light NL. The output circuit 23 generates the infrared light detection signal by performing processing such as analog-to-digital conversion on the light-receiving intensity data of the infrared light NL detected by the light-receiving element 21. The output circuit 23 generates the green light detection signal based on the received green light GL. The output circuit 23 generates the green light detection signal by performing processing such as analog-to-digital conversion on the light-receiving intensity data of the green light GL detected by the light-receiving element 21. The output circuit 23 outputs the red light detection signal, the infrared light detection signal, and the green light detection signal to the control unit 30.

The band-pass filter 25 may separate the light-receiving intensity data into the DC component data 51 and the AC component data 53. The band-pass filter 25 separates the light-receiving intensity data into the DC component data 51 and the AC component data 53 by extracting the AC component data 53 from the light-receiving intensity data. The separated DC component data 51 and the AC component data 53 are output to the control unit 30 as detection signals.

After generating the red light detection signal, the infrared light detection signal, and the green light detection signal, the measurement device 100 executes the short time Fourier transform in step S107. The control unit 30 acquires the red light detection signal, the infrared light detection signal, and the green light detection signal output from the output circuit 23. The control unit 30 executes the short time Fourier transform on the red light detection signal, the infrared light detection signal, and the green light detection signal.

The data processing unit 33 operating in the control unit 30 executes the short time Fourier transform on the red light detection signal to calculate the red light spectrogram. The data processing unit 33 executes the short time Fourier transform on the infrared light detection signal to calculate the infrared light spectrogram. The data processing unit 33 executes the short time Fourier transform on the green light detection signal to calculate the green light spectrogram. The red light spectrogram, the infrared light spectrogram, and the green light spectrogram are indicated by signal intensities of frequency components for each elapsed time.

After executing the short time Fourier transform, the measurement device 100 determines the pulse wave region PB in step S109. The data processing unit 33 determines the pulse wave region PB using the green light spectrogram obtained by the short time Fourier transform on the green light detection signal. The data processing unit 33 detects a frequency at which the signal intensity indicates a peak value. The data processing unit 33 determines the pulse wave region PB by detecting the frequency at which the signal intensity indicates a peak value. The pulse wave region PB is a region including the frequency at which the signal intensity indicates a peak value. The pulse wave region PB is, for example, a frequency range whose median is a frequency at which the signal intensity indicates a peak value. A region width of the pulse wave region PB is appropriately set in advance. The pulse wave region PB may be a frequency at which the signal intensity indicates a peak value. The green light detection signal is less susceptible to disturbance caused by body movement or the like. The peak value of the signal intensity of the green light spectrogram is likely to indicate the frequency of the pulse wave. By using the green light detection signal, the detection accuracy of the pulse wave region PB is improved.

After determining the pulse wave region PB, the measurement device 100 detects the red light detection signal intensity and the infrared light detection signal intensity in step S111. The data processing unit 33 detects the red light detection signal of the pulse wave region PB as the red light detection signal intensity. The data processing unit 33 detects the infrared light detection signal of the pulse wave region PB as the infrared light detection signal intensity.

For example, the data processing unit 33 detects, as the red light detection signal intensity, the peak value of the red light detection signal included in the pulse wave region PB. The data processing unit 33 detects, as the red light detection signal intensity, an added value of the peak value of the red light detection signal and a signal value of a frequency adjacent to the frequency indicating the peak value. The data processing unit 33 detects, as the red light detection signal intensity, a sum of the red light detection signals in the pulse wave region PB.

For example, the data processing unit 33 detects, as the infrared light detection signal intensity, the peak value of the infrared light detection signal included in the pulse wave region PB. The data processing unit 33 detects, as the infrared light detection signal intensity, an added value of the peak value of the signal intensity of the infrared light NL and a signal value of a frequency adjacent to the frequency indicating the peak value. The data processing unit 33 detects, as the infrared light detection signal intensity, a sum of the infrared light detection signals in the pulse wave region PB.

After detecting the red light detection signal intensity and the infrared light detection signal intensity, the measurement device 100 calculates the fluctuation component amplitude ratio in step S113. The data processing unit 33 calculates the fluctuation component amplitude ratio using the red light detection signal intensity and the infrared light detection signal intensity. The data processing unit 33 uses the red light detection signal intensity as an intensity of the AC component of the red light detection signal in Formula (1). The data processing unit 33 uses the infrared light detection signal intensity as an intensity of the AC component of the infrared light detection signal in Formula (1). The data processing unit 33 calculates an intensity of the DC component of the red light detection signal. The data processing unit 33 calculates an intensity of the DC component of the infrared light detection signal. The data processing unit 33 may use the DC component separated by the band-pass filter 25 as the intensity of the DC component of the detection signal.

The data processing unit 33 calculates the fluctuation component amplitude ratio using the red light detection signal intensity, the infrared light detection signal intensity, the intensity of the DC component of the red light detection signal, and the intensity of the DC component of the infrared light detection signal. The calculated fluctuation component amplitude ratio corresponds to the oxygen saturation concentration.

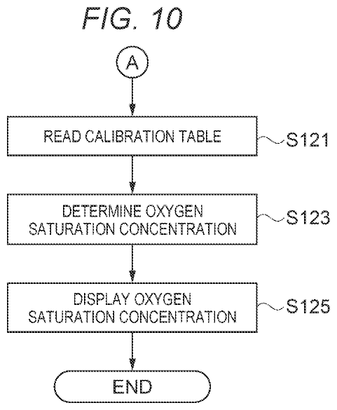

FIG. 10 shows a flowchart for determining an oxygen saturation concentration. The flowchart shown in FIG. 10 is a part of the oxygen saturation concentration measurement method. FIG. 10 shows a method of determining the oxygen saturation concentration performed by the measurement device 100. FIG. 10 shows a flowchart following FIG. 9.

After calculating the fluctuation component amplitude ratio, the measurement device 100 reads the calibration table PT in step S121. The control unit 30 reads the calibration table PT stored in the memory 40. The calibration table PT shows the relationship between the fluctuation component amplitude ratio and the oxygen saturation concentration.

After reading the calibration table PT, the measurement device 100 determines the oxygen saturation concentration in step S123. The data processing unit 33 determines, by using the calibration table PT, the oxygen saturation concentration corresponding to the fluctuation component amplitude ratio calculated in step S113. The data processing unit 33 determines the oxygen saturation concentration corresponding to the fluctuation component amplitude ratio calculated for each predetermined time interval. For example, the data processing unit 33 calculates the fluctuation component amplitude ratio and determines the corresponding oxygen saturation concentration per second.

The data processing unit 33 may calculate a moving average of the oxygen saturation concentrations determined at the predetermined time intervals. For example, the data processing unit 33 calculates a moving average of the oxygen saturation concentrations every eight seconds. The data processing unit 33 determines the oxygen saturation concentration at times T1, T2, T3, T4, T5, T6, T7, T8, and T9. T1, T2, T3, T4, T5, T6, T7, T8, and T9 have one second interval in between. The oxygen saturation concentrations determined at T1, T2, T3, T4, T5, T6, T7, T8, and T9 are indicated by D1, D2, D3, D4, D5, D6, D7, D8, and D9. When T8 elapses, the data processing unit 33 calculates an average value of D1, D2, D3, D4, D5, D6, D7, and D8 as the oxygen saturation concentration. The calculated average value is defined as the oxygen saturation concentration of T8. When T9 elapses, the data processing unit 33 calculates an average value of D2, D3, D4, D5, D6, D7, D8, and D9 as the oxygen saturation concentration. The calculated average value is defined as the oxygen saturation concentration of T9. The data processing unit 33 calculates the moving average of the oxygen saturation concentrations at eight second intervals as the oxygen saturation concentration.

The data processing unit 33 calculates the moving average of the oxygen saturation concentrations, but is not limited thereto. The data processing unit 33 may calculate a moving average of the fluctuation component amplitude ratios. The data processing unit 33 may determine the oxygen saturation concentration by referring to the moving average of the fluctuation component amplitude ratios in the calibration table PT.

After determining the oxygen saturation concentration, the measurement device 100 displays the oxygen saturation concentration in step S125. The display control unit 35 operated in the control unit 30 generates the display data including the oxygen saturation concentration and outputs the display data to the display panel 4. The display panel 4 acquires the display data and displays the oxygen saturation concentration based on the display data. The display control unit 35 causes the display panel 4 to display the oxygen saturation concentration by outputting the display data to the display panel 4. The display panel 4 displays the oxygen saturation concentrations determined at the predetermined time intervals.

The display control unit 35 may output display data including the moving average of the oxygen saturation concentrations to the display panel 4. The display panel 4 displays the moving average of the oxygen saturation concentrations as an oxygen saturation concentration. The display control unit 35 causes the display panel 4 to display the moving average of the oxygen saturation concentrations by outputting the display data including the moving average of the oxygen saturation concentrations.

The control unit 30 may transmit the oxygen saturation concentration to the external device via the communication interface. The control unit 30 may transmit various detection signals, the calculated fluctuation component amplitude ratio, or the like to the external device via the communication interface.

The measurement device 100 includes: the light-emitting element unit 10 including the red light-emitting element 11a that emits the red light RL, the infrared light-emitting element 11b that emits the infrared light NL, and the green light-emitting element 11c that emits the green light GL; the light-receiving element unit 20 that receives the red light RL, the infrared light NL, and the green light GL emitted by the light-emitting element unit 10 and generates the red light detection signal based on the red light RL, the infrared light detection signal based on the infrared light NL, and the green light detection signal based on the green light GL; and the control unit 30 that calculates the biological information based on the red light detection signal, the infrared light detection signal, and the green light detection signal. The control unit 30 performs the short time Fourier transform on the red light detection signal, the infrared light detection signal, and the green light detection signal, determines the pulse wave region PB including the frequency of the pulse wave by the short time Fourier transform on the green light detection signal, detects the red light detection signal intensity of the pulse wave region PB and the infrared light detection signal intensity of the pulse wave region PB, and calculates the fluctuation component amplitude ratio between the red light transmitted amount and the infrared light transmitted amount using the red light detection signal intensity and the infrared light detection signal intensity.

Since the green light detection signal based on the green light GL is hardly affected by body movement of the user M, the outside air temperature, or the like, the pulse wave component is easily detected. By determining the pulse wave region PB using the green light detection signal, the red light detection signal intensity and the infrared light detection signal intensity are easily specified. The measurement accuracy of the fluctuation component amplitude ratio is less likely to decrease.

The measurement device 100 includes the memory 40 that stores the calibration table PT indicating the relationship between the fluctuation component amplitude ratio and the oxygen saturation concentration. The control unit 30 determines the oxygen saturation concentration as the biological information based on the calculated fluctuation component amplitude ratio.

Since the measurement accuracy of the fluctuation component amplitude ratio is less likely to decrease, the measurement accuracy of the oxygen saturation concentration determined based on the fluctuation component amplitude ratio is improved.

The measurement device 100 includes the display panel 4 that displays the oxygen saturation concentration. The control unit 30 causes the display panel 4 to display the oxygen saturation concentration.

The user M can confirm the oxygen saturation concentration at which a decrease in measurement accuracy is small.

The control unit 30 determines the oxygen saturation concentration at a predetermined time interval, calculates a moving average of the plurality of oxygen saturation concentrations determined at the predetermined time intervals, and causes the display panel 4 to display the moving average of the oxygen saturation concentrations.

The oxygen saturation concentration in which variation in the measurement result due to an influence of noise is prevented is displayed.

The light-receiving element unit 20 receives the red light RL, the infrared light NL, and the green light GL reflected by the user M.

Since the reflected light can be measured, the oxygen saturation concentration can be measured at a site other than a fingertip.

The oxygen saturation concentration measurement method includes: emitting the red light RL, the infrared light NL, and the green light GL to the user M; receiving the red light RL, the infrared light NL, and the green light GL passing through the user M; generating the red light detection signal based on the received red light RL, the infrared light detection signal based on the received infrared light NL, and the green light detection signal based on the received green light GL; performing the short time Fourier transform on the red light detection signal, the infrared light detection signal, and the green light detection signal; determining the pulse wave region PB including the frequency of the pulse wave by the short time Fourier transform on the green light detection signal; detecting the red light detection signal intensity of the pulse wave region PB and the infrared light detection signal intensity of the pulse wave region PB; and calculating the fluctuation component amplitude ratio between the red light transmitted amount and the infrared light transmitted amount using the red light detection signal intensity and the infrared light detection signal intensity.

By determining the pulse wave region PB using the green light detection signal, the red light detection signal intensity and the infrared light detection signal intensity are easily specified. The measurement accuracy of the fluctuation component amplitude ratio is less likely to decrease.

The oxygen saturation concentration measurement method includes: determining the oxygen saturation concentration as the biological information based on the calculated fluctuation component amplitude ratio and the calibration table PT indicating the relationship between the fluctuation component amplitude ratio and the oxygen saturation concentration.

Since the measurement accuracy of the fluctuation component amplitude ratio is less likely to decrease, the measurement accuracy of the oxygen saturation concentration determined based on the fluctuation component amplitude ratio is improved.

What is claimed is:

1. A biological information measurement device comprising:
a light-emitting unit including a first light-emitting element that emits red light, a second light-emitting element that emits infrared light, and a third light-emitting element that emits green light;
a light-receiving unit configured to receive the red light, the infrared light, and the green light emitted by the light-emitting unit, and generate a first light-receiving signal based on the red light, a second light-receiving signal based on the infrared light, and a third light-receiving signal based on the green light; and
a controller configured to calculate biological information based on the first light-receiving signal, the second light-receiving signal, and the third light-receiving signal, wherein
the controller is configured to
perform time-frequency analysis on the first light-receiving signal, the second light-receiving signal, and the third light-receiving signal,
determine a pulsation band including a frequency of a pulse wave by the time-frequency analysis on the third light-receiving signal,
detect a first light-receiving signal intensity of the pulsation band and a second light-receiving signal intensity of the pulsation band, and
calculate a fluctuation component ratio between a red light transmitted amount and an infrared light transmitted amount by using the first light-receiving signal intensity and the second light-receiving signal intensity.

2. The biological information measurement device according to claim 1, further comprising:
a storage unit configured to store a calibration curve table indicating a relationship between the fluctuation component ratio and an oxygen saturation concentration, wherein
the controller is configured to determine the oxygen saturation concentration as the biological information based on the calculated fluctuation component ratio.

3. The biological information measurement device according to claim 2, further comprising:
a display unit configured to display the oxygen saturation concentration, wherein
the controller is configured to cause the display unit to display the oxygen saturation concentration.

4. The biological information measurement device according to claim 3, wherein
the controller is configured to
determine the oxygen saturation concentration at a predetermined time interval,
calculate a moving average of the plurality of oxygen saturation concentrations determined at the predetermined time interval, and
cause the display unit to display the moving average of the oxygen saturation concentrations.

5. The biological information measurement device according to claim 1, wherein
the light-receiving unit is configured to receive the red light, the infrared light, and the green light reflected by a subject.

6. A biological information measurement method comprising:
emitting red light, infrared light, and green light to a subject;
receiving the red light, the infrared light, and the green light passing through the subject;
generating a first light-receiving signal based on the received red light, a second light-receiving signal based on the received infrared light, and a third light-receiving signal based on the received green light;
performing time-frequency analysis on the first light-receiving signal, the second light-receiving signal, and the third light-receiving signal;
determining a pulsation band including a frequency of a pulse wave by the time-frequency analysis on the third light-receiving signal;
detecting a first light-receiving signal intensity of the pulsation band and a second light-receiving signal intensity of the pulsation band; and
calculating a fluctuation component ratio between a red light transmitted amount and an infrared light transmitted amount by using the first light-receiving signal intensity and the second light-receiving signal intensity.

7. The biological information measurement method according to claim 6, further comprising:
determining, based on the calculated fluctuation component ratio and a calibration curve table indicating a relationship between the fluctuation component ratio and an oxygen saturation concentration, the oxygen saturation concentration as biological information.
